(19) United States
(12) Patent Application Publication
(10) Pub. No.: US 2014/0309713 A1
(43) Pub. Date: Oct. 16, 2014

(54) LONG-TERM ELECTROSTIMULATION OF RECEPTORS

(71) Applicants: Ronnie Levy, Kochav-Yair (IL); Yiftach Beinart, Hod Hasharon (IL); Alon Shalev, Ra'anana (IL)

(72) Inventors: Ronnie Levy, Kochav-Yair (IL); Yiftach Beinart, Hod Hasharon (IL); Alon Shalev, Ra'anana (IL)

(21) Appl. No.: 13/862,428

(22) Filed: Apr. 14, 2013

(51) Int. Cl. A61N 1/36; A61N 1/372

(52) U.S. Cl. CPC A61N 1/36057; A61N 1/37235; A61N 1/36135
USPC 607/59; 607/60

(57) ABSTRACT

An electrostimulation device includes an implantable internal unit, which includes an array of electrodes assembled on or in a housing, and affixing structure for affixing the unit with respect to a targeted anatomy of a patient, a microprocessor mounted in the housing and in communication with the electrodes, a transceiver mounted in the housing and in communication with the microprocessor, and a power supply for providing power to the electrodes, the microprocessor and the transceiver.

### FIELD OF THE INVENTION

The present invention relates generally to electro stimulation of receptors, such as chemoreceptors, baroreceptors and aortic arch receptors, such as for inducing changes in the diameter of blood vessels of the brain, including dilation and constriction.

### BACKGROUND OF THE INVENTION

The cardiovascular center of the brain includes groups of neurons scattered within the medulla of the brain stem, which regulate heart rate, contractility of the ventricles, and blood vessel diameter. The cardiovascular center receives input both from higher brain regions and from sensory receptors. The two main types of sensory receptors that provide input to the cardiovascular center are baroreceptors and chemoreceptors. Baroreceptors are pressure-sensitive sensory neurons that monitor stretching of the walls of blood vessels and the atria.

The carotid body is a small cluster of chemoreceptors and supporting cells located near the bifurcation of the carotid artery (where it bifurcates to the internal and external carotid arteries), for both the left and right sides of the neck. Chemoreceptors monitor blood acidity (PH), partial pressure of oxygen and of carbon dioxide. Each carotid body is a few millimeters in size and has the distinction of having the highest blood flow per tissue weight of any organ in the body.

PCT Patent Application PCT/IL2012/000290, filed 2 Aug. 2012, describes stimulation of chemoreceptors and baroreceptors in a carotid artery. In one embodiment, a device is inserted intravascularly via the femoral artery. In another embodiment, a device is introduced in an extravascular approach.

### SUMMARY

The present invention seeks to provide a long-term implantable device for electrostimulation of receptors. Embodiments of the invention can be used to stimulate the carotid sinus nerve, aortic nerve, chemoreceptors adjacent to the bifurcation of the carotid, baroreceptors adjacent to the bifurcation of the carotid, aortic arch chemoreceptors and aortic arch baroreceptors, and others. In other words, the invention can be used to modulate the activity of the chemoreceptors to increase (stimulate) or decrease (inhibit) their activity and as a consequence to cause changes in the diameter of blood vessels of the brain, including dilation and constriction.

The device of the present invention is a long term implantable device, which can be used to treat various symptoms, disorders and diseases, such as but not limited to, those related to the central nervous system (e.g., migraines, dementia and many others), CHF (chronic heart failure) or other kinds of heart failure, hypothermia, diabetes, COPD (chronic obstructive pulmonary disease), cerebral brain vasospasm, ischemia, brain injury and many others.

In one embodiment, the device of the invention includes an implantable electrostimulation device (internal device) and an external controller that controls operating parameters of the internal device. The external controller may be used by the physician near the patient or may be used from a remote site. The internal device may be introduced percutaneously (intravascular or extravascular) or by surgery. A power supply, such as but not limited to, rechargeable batteries, may be provided with the internal device or may be external to the patient, such as with the external controller.

### BRIEF DESCRIPTION OF THE DRAWINGS

The present invention will be understood and appreciated more fully from the following detailed description, taken in conjunction with the drawings in which:

FIG. 1 is a simplified illustration of an electrostimulation device, constructed and operative in accordance with a non-limiting embodiment of the present invention; and

FIG. 2 is a simplified illustration of the electrostimulation device implanted in a neck of a patient with electrodes positioned near the carotid bifurcation, in accordance with a non-limiting embodiment of the present invention.

### DETAILED DESCRIPTION

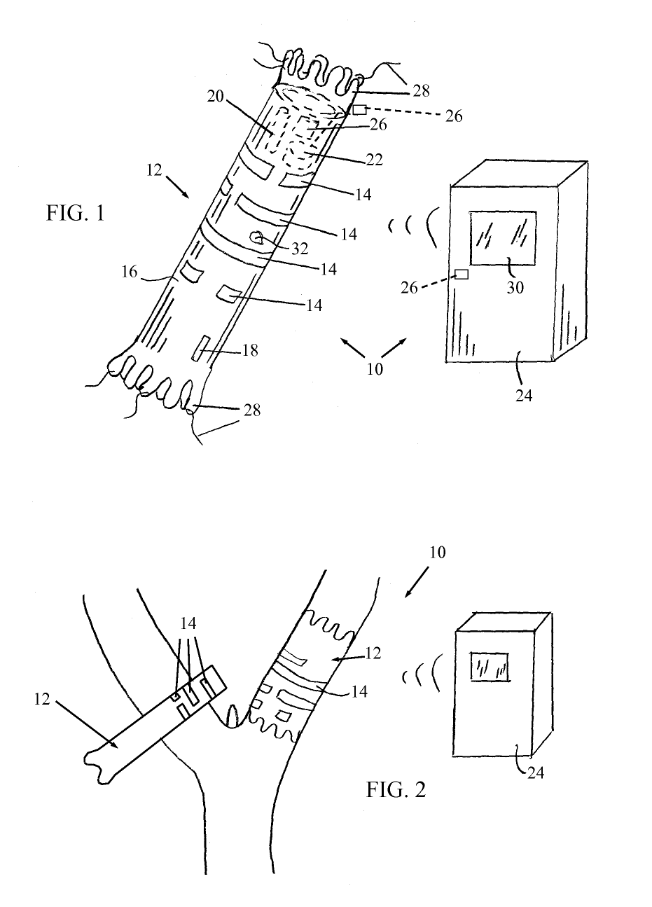

Reference is now made to FIG. 1, which illustrates an electrostimulation device 10, constructed and operative in accordance with a non-limiting embodiment of the present invention.

Electrostimulation device 10 includes an implantable internal unit 12, which includes an array of electrodes 14 assembled on or in a housing 16. Electrodes 14 extend at least partially around a perimeter of housing 16 and can be of any size and shape. In one embodiment, housing 16 is cylindrical, and electrodes 14 are full 360° rings around the circumference of housing 16 (an example of which is the third lowest electrode in the figure). In another embodiment, electrodes are partial rings that do not extend completely 360° around the circumference of housing 16 (examples of which are the two upper electrodes in the figure). In another embodiment, electrodes are discrete non-ring members (an example of which is the lowest electrode in the figure). The electrodes 14 are of course insulated from one another, either by the housing being made of an electrically insulating material or by placing the electrodes on a substrate made of an electrically insulating material. One or more fiducial markers 18, which may be radiopaque, are preferably disposed on or in housing 16 to aid in positioning the unit 12 in its proper position with respect to the targeted anatomy (carotid body in this example).

Electrodes 14 may be communicate (in wired or wireless connection) with a microprocessor 20, which is also mounted in housing 16. Microprocessors of small size, such as but not limited to, a diameter of 2 mm and length of 15 mm, are technologically achievable. A transceiver 22 mounted in housing 16 is also connected to microprocessor 20. Transceiver 22 communicates with an external controller 24 that controls operating parameters of the electrodes 14. A power supply 26, such as but not limited to, rechargeable batteries, may be provided for providing power to the internal unit 12. Power supply 26 may be internal to housing 16, or may be external to housing 16 (e.g., connected to internal components by wire, as shown in broken lines in FIG. 1) or may be external to the patient, such as with the external controller 24 (in which case the power is transmitted wirelessly or by wired connection).

Implantable internal unit 12 includes affixing structure 28 for affixing the unit with respect to (e.g., affixing in, on, near or to) the targeted anatomy, that is, tissue or blood vessels near the targeted anatomy. Without limitation, affixing structure 28 includes expandable stent like structure (e.g., balloon expandable or self-expandable, shape memory alloys that expand at the installation site), sutures, loops, barbs or a combination thereof.

One or more sensors 32 may be provided for sensing biological information, which may be transmitted via transceiver 22 to external controller 24 so as to operate device 10 in a closed control loop. Sensors 32 may include, without limitation, chemical sensors, pH sensors, temperature sensors, and others.

The internal unit 12 may be fully autonomic and operate without the need of the external controller 24.

External controller 24 controls operating parameters associated with energization of electrodes 14, such as current and frequency of signals used to energize the electrodes, shape and strength of the electrical field, and others. The electrodes 14 may be used in bipolar mode, and optionally in monopolar mode or a combination thereof. The operator (such as the physician) selects the desired operating parameters with a data interface 30 on external controller 24. The data is sent, preferably wirelessly, to transceiver 22, which deliver the data to microprocessor 20, which activates the electrodes 14.

Reference is now made to FIG. 2, which illustrates the implantable internal unit 12 of the electrostimulation device 10 implanted in a neck of a patient. The electrodes 14 are positioned near the carotid bifurcation. The internal unit 12 may be introduced percutaneously, such as by an intravascular route, and affixed by affixing structure 28 in the external carotid artery extending from the common carotid artery (of course, it could alternatively be affixed in the internal carotid artery). Alternatively, internal unit 12 may be introduced by an extravascular route, percutaneously or by any suitable surgical method or any combination of percutaneous and surgical intervention and affixed to neighboring tissue or bone as shown in FIG. 2 (e.g., crossing near the internal carotid artery).

The electric stimulation can be optimized by external controller 24 to modulate receptors, such as chemoreceptors, baroreceptors and aortic arch receptors, such as to increase (stimulate) or decrease (inhibit) their activity, so as to cause changes in the desired physiological response, such as diameter of blood vessels of the brain, including dilation and constriction.

The electrodes can be activated in any combination and in any order. The combinations and order can be changed during a stimulation session, either as part of a pre-determined sequence or in response to feedback from the patient.

The signal profile used to energize the electrodes can be of a wide variety—burst, prolonged, intermittent and any combination thereof. Individual groups of signals, such as but not limited to individual bursts, can have a step profile, a ramped profile that increases monotonically from the beginning to the end of the group of signals, a ramped profile that decreases monotonically from the beginning to the end of the group, a ramped profile which increases from a small value to a predetermined value, then remains constant until the end of the group, a ramped profile that starts at a predetermined value, remains at that value for a predetermined portion of the group, then decreases to a small value at the end of the group, a sinusoidal signal profile, a triangular signal profile, and any combination thereof.

Dipole stimulation of the receptors or neurons is carried out by rapidly changing the electrical field around the electrodes 14. The waveform of the electrical signal significantly affects the threshold of energy applied to the receptors. The longitudinal component of the electric field excites the nerve, so the current lines should be along the nerve's longitudinal axis; in other words, the electric field should be optimally implemented so that the longitudinal vectors are along the carotid body region. Balance biphasic waveforms are preferred because the equivalent charge is neutral and thus reduces possible tissue damage. The electric field should be localized and balanced as much as possible (longitudinally and radially) and its amplitude should be as low as possible in order to reduce possible side effects, such as other physiological effects, and tissue damage.

1. An electrostimulation device comprising:
an implantable internal unit, which comprises an array of electrodes assembled on or in a housing, and affixing structure for affixing said unit with respect to a targeted anatomy of a patient;
a microprocessor mounted in said housing and in communication with said electrodes;
a transceiver mounted in said housing and in communication with said microprocessor; and
a power supply for providing power to said electrodes, said microprocessor and said transceiver.

2. The electrostimulation device according to claim 1, further comprising an external controller operable to communicate with said transceiver and to monitor and control operating parameters of said electrostimulation device.

3. The electrostimulation device according to claim 1, further comprising one or more sensors operative to sense biological information.

4. The electrostimulation device according to claim 2, further comprising one or more sensors operative to sense biological information, said one or more sensors being in communication via said transceiver with said external controller.

5. The electrostimulation device according to claim 1, wherein said power supply is internal to said implantable internal unit.

6. The electrostimulation device according to claim 1, wherein said power supply is external to said implantable internal unit.

7. The electrostimulation device according to claim 1, wherein said affixing structure comprises expandable stent-like structure.

8. The electrostimulation device according to claim 1, wherein said affixing structure comprises sutures.

9. The electrostimulation device according to claim 1, wherein said electrodes extend at least partially around a perimeter of said housing.

10. The electrostimulation device according to claim 1, further comprising one or more fiducial markers disposed on or in said housing.

11. A method for electrostimulation comprising:
implanting at least one electrostimulation device of claim 1 into a patient and energizing said electrodes to cause neurostimulation.
